(19) United States
(12) Patent Application Publication
(10) Pub. No.: US 2013/0266637 A1
(43) Pub. Date: Oct. 10, 2013

(54) COMPOSITION FOR TREATING CANCER BY THE CONTROLLED RELEASE OF AN ACTIVE SUBSTANCE

(71) Applicants: Holger Egger, Koln (DE); Bastian Budde, Koln (DE); Kai Termming, Leverkusen (DE); Anna Arbuzova, Berlin (DE); Andrea Gramatica, Berlin (DE); Andreas Herrmann, Berlin (DE); Juergen Liebscher, Berlin (DE); Nicolai Brodersen, Berlin (DE)

(72) Inventors: Holger Egger, Koln (DE); Bastian Budde, Koln (DE); Kai Termming, Leverkusen (DE); Anna Arbuzova, Berlin (DE); Andrea Gramatica, Berlin (DE); Andreas Herrmann, Berlin (DE); Juergen Liebscher, Berlin (DE); Nicolai Brodersen, Berlin (DE)

(73) Assignee: BAYER INTELLECTUAL PROPERTY GMBH, MONHEIM (DE)

(21) Appl. No.: 13/878,801

(22) PCT Filed: Oct. 11, 2011
(86) PCT No.: PCT/EP11/67702

§ 371 Date: Jun. 26, 2013

(30) Foreign Application Priority Data
Oct. 12, 2010 (DE) 102010042338.6

(51) Int. Cl. A61K 47/22

(52) U.S. Cl. CPC A61K 47/22
USPC 424/450; 514/34

(57) ABSTRACT

The invention relates to a novel composition for treating cancer, which allows the active substance to be released in the direct presence of the cells which are to be treated with the active substance. Said composition comprises an active substance carrier which is in the form of a liposome comprising a disulfide group, a cytostatic agent being contained in the active substance carrier. The invention also relates to a method for the locally defined release of a cytostatic agent.

The present invention relates to a new type of composition for treating cancer which allows the active ingredient to be released in the direct presence of the cells to be treated with the active ingredient. The composition comprises an active ingredient carrier in the form of a liposome comprising a disulfide group, a cytostatic agent being contained in the active ingredient carrier. The invention likewise relates to a method for the locally defined release of a cytostatic agent.

Carrier systems for active ingredient release based on liposome carriers in the magnitude of about 100 nm are generally known in the meantime in medical technology. They are generally considered to be advantageous if a substance that is toxic for certain organs but which can be used at other sites in the human body as a medicinal active ingredient is to be released only at those desired sites.

The aforementioned toxic substances also include so-called cytostatic agents which, however, in the correct dosage and when used on certain, especially degenerately diseased tissue types, can also be used in medical therapeutics.

The aforementioned liposome carriers are generally understood as meaning a substance group which consists of nontoxic phospholipids. Such liposome carriers often also include polymers, such as, for example, polyethylene glycols, which are characterized by improved stability in the bloodstream of treated patients, since the aforementioned polyethylene glycols form a form of steric protection around the actual liposome carriers. This gives so-called lipopolymers.

The above statements relating to liposome carriers are also confirmed by J. Davidsen et al. in “Secreted phospholipase A2 as a new enzymatic trigger mechanism for localised liposomal drug release and absorption in diseased tissue”, published in Biochimica et Biophysica Acta 1609 (2003) 95-101. Furthermore, J. Davidsen et al. disclose that the properties of such active ingredient carriers in the form of the aforementioned liposome carriers can depend, especially with regard to their release behavior, on a multitude of chemical and physical parameters, the interplay of which is still not completely understood.

J. Davidsen et al. disclose in this connection their investigations regarding a liposome formulation consisting of a liposome carrier measuring 100 nm across. The liposome carrier can consist of 1,2-hexadecanoyl-sn-glycero-3-phosphocholine (DPPC) and/or 1,2-hexadecanoyl-sn-glycero-3-phosphoethanolamine-N-[methoxy(polyethylene glycol)-2000] (DPPE-PEG2000) and/or 1-O-hexadecanoyl-2-hexanodecanoyl-sn-glycero-3-phosphocholine (1-O-DPPC) and/or 1-O-hexadecanoyl-2-hexanodecanoyl-sn-glycero-3-phosphoethanolamine-N-[methoxy(polyethylene glycol)-350] (1-O-DPPE-PEG350). The aforementioned substances are referred to as lipids which can also form multilamellar vesicles and which belong to the substance group of the phospholipids.

It is also disclosed that calcein can be included in the liposome formulation comprising 1-O-DPPC and 10 mol % of 1-O-DPPE-PEG350. Calcein is referred to here as a model substance for drugs. A release of the calcein can be achieved, according to the disclosure by J. Davidsen et al., by means of treating this liposome formulation with phospholipase A2 (PLA2). The release takes place by the hydrolytic cleavage of either DPPC or 1-O-DPPC, or 1-O-DPPE-PEG350, which is catalyzed by PLA2, giving a lysolipid and a fatty acid. The liposome formulations disclosed by J. Davidsen are only described in connection with the aforementioned model substances as regards their release. J. Davidsen also describe an option of using liposome formulations in general for the release of doxorubicin, which is a cytostatic agent. However, no actual disclosure regarding this fact is given.

Consequently, J. Davidsen et al. disclose neither a cleavage into a hydrophobic and a hydrophilic moiety, nor that the liposome carrier can comprise a disulfide bond. In particular, J. Davidsen et al. do not disclose that a cytostatic agent is released in the course of this.

In “Reduction-sensitive polymers and bioconjugates for biomedical applications”, published in Biomaterials 30 (2009) 2180-2198, Meng et al. disclose that certain polymers with disulfide bridges are suitable for incorporating active ingredients either in the polymeric strand or in one of its side chains. According to Meng et al., the active ingredients used here also include cancer drugs. However, the substances disclosed therein always have a polymeric character since particles, i.e. solids, are formed from these into which the potential active ingredients are to be incorporated.

These are therefore in no case substances with a lipid character which would be suitable for producing liposomes.

The disclosed mode of action of the substances is based on the possibility of the bond cleavage of the disulfide group as a result of which either the polymers are eliminated or the polyethylene glycol side chains which are bonded with the disulfide group to the polymers are separated off and therefore the particle, after being used in the patient, can be recognized and broken down by their immune system. Accordingly, the release from the active ingredient carrier is based, according to the disclosure by Meng et al., either on an elimination of a solid as a result of the cleavage of polymeric bonds or as a result of degradation of the solid by the immune system of the patient after the particle has been recognized for this. In no case is the active ingredient released directly.

WO 2000/059474 A1 discloses lipid compositions which consist of a hydrophobic “tail group” and a hydrophilic “head group”, the last-mentioned hydrophilic “head group” being covalently bonded to the hydrophobic “tail group”. Within the hydrophilic “head group”, a distinction is made between a first and a second region, these two regions being joined by a disulfide group, which can be cleaved, for example, by glutathione. The first region of the hydrophilic “head group” carries, according to WO 2000/059474 A1, a positive charge at a physiological pH and the second region carries a negative charge at a physiological pH. The first region is covalently bonded to the hydrophobic “tail group”.

The chemical formula of the aforementioned lipid compositions disclosed in general terms according to WO 2000/059474 A1 is X—Y—S—S—Z. Here, the chemical group X forms the aforementioned hydrophobic “tail group” and Y, and also Z form the first or second region of the hydrophilic head group.

According to the further disclosure of WO 2000/059474 A1, in the aforementioned chemical formula, the chemical group X as hydrophobic “tail group” can be a group which is essentially similar to the glycerol trunk of a fat and to which the chemical group Y is bonded via a further chemical group W.

The chemical group W can be selected from the list consisting of CHR₃, NR₃, N⁺(R₃)₂, O, S, C(O)NH, NH(CO), OC(O)NH and OP(O)(OR₃)O. The chemical group R3 also present in the chemical group W can be hydrogen or a C₁-C₄ alkyl radical.

The chemical group Y can be a C₁-C₁₂ alkyl radical, a C₂-C₁₂ alkenyl radical or a C₂-C₁₂ alkynyl radical with substituents in the form of alkyl radicals, amino radicals, aminoalkyl radicals, guanidine radicals, guanidine alkyl radicals, amido radicals or amidoalkyl radicals, in which case the aforementioned alkyl, alkenyl, or alkynyl radicals of the chemical group Y can be further interrupted by NR₃, N⁺(R₃)₂, C(O), NHC(NH), C(NH)NH, NHC(NH)NH. Alternatively the chemical group Y can also be an amino acid radical or a peptide.

According to the disclosure of WO 2000/059474 A1, the chemical group Z can be a C₁-C₁₂ alkyl radical, alkenyl radical or an alkynyl radical, which in turn can be substituted with alkyl radicals, carboxyl radicals, carboxyalkyl radicals, amino acid radicals, peptides, oligonucleotides or so-called “target molecule radicals”.

However, the aforementioned chemical groups X, Y and Z must be configured in each case such that the groups X and Y in their totality have a positive charge at a physiological pH and the group Z has a negative charge at a physiological pH.

Consequently, cleaving substances of the formula X—Y—S—S—Z at the disulfide group according to WO 2000/059474 A1 produces a group of the type X—Y—S, which is positively charged, and a chemical group of the type S—Z, which is negatively charged.

In each case, the group of the type X—Y—S, as a result of the fact that it always still comprises the “hydrophobic tail group” and a positive charge, is a chemical group which has amphiphilic properties, i.e. which is hydrophobic in one region and hydrophilic in another region. The group of the type S—Z, as a result of its negative charge and as a result of the fact that it comprises a C₁-C₁₂ alkyl radical, alkenyl radical or an alkynyl radical, is either likewise amphiphilic or even—in the case of particularly short alkyl radicals, alkenyl radicals or alkynyl radicals—only hydrophilic.

WO 2000/059474 A1 further discloses that the aforementioned substances are suitable for producing liposome carriers by means of which a targeted release of substances can take place at a site of action. The release is based on the chemical cleavage of the disulfide group. WO 2000/059474 A1 also discloses the use of the liposome carriers therein for releasing cancer drugs in general.

However, WO 2000/059474 A1 does not disclose that a cleavage of a previously amphiphilic substance into a hydrophilic moiety and a hydrophobic moiety can be achieved.

However, such a division into a hydrophilic and a hydrophobic moiety would allow a particularly efficient destabilization and elimination of the liposome carrier constructed with such substances.

Besides the compositions described above, it is also generally known that specific enzymes in the body of a mammal attack specific bonds of substances and cleave them. In this connection, for example, D. Mustacich et al. disclose in Biochem. J. (2000) 346, 1-8, “Thioredoxin reductase”, that such an enzyme is thioredoxin reductase (abbreviated to TrxRs). It is also disclosed that this enzyme is present especially in tumor tissues in at least ten times the amount compared to in healthy tissue.

The cytostatic agents used in the treatment of cancer diseases are not selective as regards their cytostatic effect. Accordingly, this effect is generally developed both towards healthy cells and also towards the diseased tissue. The cytostatic effect on the healthy cells is a serious side effect of these active ingredients.

In the course of such treatments, the patient is often also treated with a larger dose of the cytostatic agents than the actual tumor disease requires since it has to be ensured that the amount of active ingredient administered to the patient reaches the site of action (the tumor) still in a sufficiently large dose.

For this purpose, the cytostatic side effect on the healthy cells is included in the calculation when determining the necessary dose insofar as it is assumed that some of the cytostatic active ingredient develops its effectiveness towards healthy cells and is thus no longer available for the effect against the tumor tissue. Accordingly, the aforementioned side effects are an intrinsic part of the treatment in such cases.

However, it would be desirable if the active ingredient were only released at the site of action and would only develop its effect there. This would result firstly in a reduction in the dosage and secondly a lesser, negative adverse effect on the patient.

However, in many cases it must at the same time be ensured that the release of the active ingredient at the potential site of action takes place immediately and as quantitatively as possible and not in an insidious manner.

However, the aforementioned problems have hitherto only been solved with deficiencies, if at all, in the aforementioned prior art.

It is therefore the object to provide an active ingredient carrier with a cytostatic agent present therein which can be cleaved e.g. enzymatically at a disulfide group, present in this active ingredient carrier, such that a rapid and as complete a release as possible of this cytostatic agent at the site of action is possible.

The object also consists in providing a method for releasing a cytostatic agent suitable for the targeted treatment, for example of tumor diseases, in which the cytostatic agent is released rapidly and as quantitatively as possible only in the direct vicinity of the site of action in the human body.

As a first subject matter of the present invention, a composition has been found comprising an active ingredient carrier in the form of a liposome which comprises a cytostatic agent, characterized in that the active ingredient carrier comprises at least one substance according to formula (I)

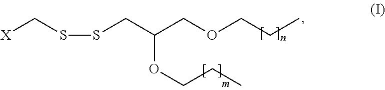

where the at least one substance according to formula (I)
(a) has in X a chemical group comprising at least five carbon atoms and comprising at least one ether group and/or amine group or ammonium group, and where
(b) n and m, independently of the other, is a natural number from 1 to 30, but where the sum of n and m is at least 16.

As a result of the property of the substances according to formula (I) in the composition according to the invention that they have in the radicals having 1 to 30 carbon atoms chemical groups which have hydrophobic properties and at the same time sterically lead to the fact that when the substances according to formula (I) are incorporated into a polar medium, these substances arrange themselves such that these hydrophobic chemical groups are arranged inwards, away from the polar solvent, these form, optionally with further substances of identical or similar properties, liposomes when incorporated into polar solvents such as, for example, water.

Accordingly, the incorporation of the substances according to formula (I) into a polar medium leads to these substances arranging themselves such that it results in the formation of spherical structures in the form of liposomes.

Here, usually so-called lipid double layers are formed, where the hydrophilic chemical groups X of the substances according to formula (I) orient themselves both outside and inside towards the polar solvent, which can also be located in the interior of the aforementioned spherical structures, whereas the hydrophobic chemical groups of both layers are oriented into the interior of the lipid double layer. In the interior of the liposome there is a cytostatic agent, dissolved in the polar medium, which, provided the lipid double layer is stable, cannot leave this and thus can also not develop a cytostatic effect towards cells located in the vicinity of the composition.

At the same time, the substances according to formula (I) in the composition according to the invention, however, can be selectively cleaved at the provided disulfide group such that, as a result, a hydrophilic chemical group consisting of the chemical group X and a sulfur radical, and also a hydrophobic chemical group comprising the radicals having 1 to 30 carbon atoms are formed. This results in an immediate and complete destabilization of the aforementioned liposome and thus in an immediate release of the cytostatic agent present.

Consequently, with the new type of composition according to the invention it is possible for the first time to release a cytostatic agent by the cleavage of a disulfide bond and to give a hydrophilic and a hydrophobic radical, which permits, depending on this, the cytostatic agent to be released spontaneously and only at the desired site of action.

The radicals with n, m carbon atoms according to formula (I) are usually saturated hydrocarbon radicals, as is also shown in formula (I).

The radicals with n, m carbon atoms can also be mono- or polyunsaturated radicals. The radicals with n, m carbon atoms can thus also have one or more double and/or triple bonds.

According to the present invention, formula (I) is accordingly not to be understood as regards the hydrocarbon radicals having n, m carbon atoms shown therein as meaning that these radicals are exclusively saturated hydrocarbon radicals having n, m carbon atoms.

In preferred embodiments, the chemical group X comprises at least eight carbon atoms.

It is likewise preferred if the chemical group X also comprises at least one carboxylic acid ester group as well as the at least one ether group and/or amine group or ammonium group.

It is particularly preferred if the chemical group X comprises either at least two ether groups or at least one amine group or ammonium group and at least two carboxylic acid ester groups.

It is likewise particularly preferred if the at least one amine group or ammonium group of the chemical group X of the substances according to formula (I) in the active ingredient carriers of the composition according to the invention is a quaternary ammonium group.

In such embodiments, the substance according to formula (I) in the composition according to the invention carries at least one positive charge and is present in combination with at least one singly or multiply negatively charged counterion. Such counterions can be for example halogen ions, such as chloride, bromide and/or iodide, but can also be any other desired counterions with negative charges.

It is very particularly preferred if the chemical group X of the substances according to formula (I) in the composition according to the invention comprises at least three ether groups. In this connection, it is preferred if the chemical group X is bonded via a carboxylic acid ester group to a chemical group Y according to the other preferred embodiment described below.

Within this very particularly preferred embodiment of the substances according to formula (I) in the composition according to the invention, the chemical group X, which then preferably further comprises only one amine group or ammonium group, is likewise preferably bonded to the radical of the substance according to formula (I) via this amine group or ammonium group.

This embodiment is particularly advantageous because the amine group or ammonium group and the at least three ether groups in their totality result in a particularly hydrophilic property of the chemical group X.

In a further preferred embodiment of the composition according to the invention, the substances according to formula (I) in the chemical group X also comprise a phosphate group.

In preferred embodiments of the composition according to the invention, in this composition, the active ingredient carrier in the form of a liposome which comprises a cytostatic agent can also comprise at least one substance according to formula (I*)

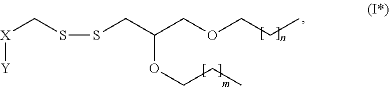

where the chemical group Y is a chemical group selected from the list consisting of biotin, protein, peptide, glyco group, nucleic acid, nucleoside or nucleotide and where X, n and m have the meanings as in formula (I) or in the preferred variants described above.

The chemical group Y in the preferred embodiment of the present invention is biotin.

In the preferred embodiment in which the chemical group Y is biotin, this can either be bonded to the chemical group X by an amide bond or via a carboxylic acid ester group.

In this preferred embodiment of the composition comprising active ingredient carrier with substances according to formula (I*), the chemical group Y can be present bonded to the chemical group X terminally or as a side group.

In all embodiments of the invention comprising substances according to formula (I*), the chemical group Y serves for (bio)chemical identification.

By identifying the group Y for example in the case of the use of biotin according to the preferred embodiment, (bio)chemically clearly by streptavidin, a further chemical and/or biological group can bind to this which imparts further (bio)chemical properties to the substance according to formula (I*).

Thus, for example, an antibody conjugated with streptavidin can be bonded to the biotin. Alternatively, also only streptavidin can be bonded to the biotin and an antibody conjugated in turn with biotin can in turn be bonded to the streptavidin. In both cases, this makes it possible for the active ingredient carrier to then accumulate preferentially at target molecules of the cells to be treated via the selective effect of the antibody.

In a further preferred embodiment of the composition according to the invention, it comprises active ingredient carriers with a substance according to formula (I*) in which Y is biotin to which streptavidin is present in bonded form, and onto which streptavidin in turn is bonded an antibody conjugated with biotin.

Particularly preferred active ingredient carriers are those comprising substances according to formula (Ia)

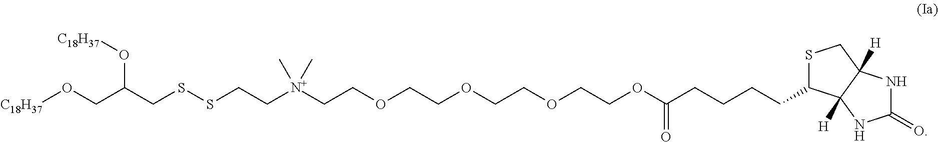

In these particularly preferred compositions with active ingredient carriers comprising substances according to formula (Ia), streptavidin is likewise preferably bonded to the biotin of the substance according to formula (Ia), onto which streptavidin is in turn bonded an antibody conjugated with biotin.

The aforementioned composition according to the invention usually comprises, in the active ingredient carrier, besides the substances according to formula (I), (I*) and/or (Ia) at least one further substance with at least one chemical group having lipophilic properties and at least one chemical group having hydrophilic properties. Such further substances are preferably lipids.

Such lipids are usually those from the substance class of phospholipids, in particular phosphatidylcholine and/or phosphatidylethanolamine.

Preferred lipids are those selected from the list consisting of L-α-phosphatidylcholine, 1,2-dioleoyl-sn-glycero-3-phosphoethanolamine, ammonium(1,2-dipalmitoyl-sn-glycero-3-phosphoethanolamine-N-lissamine rhodamine B sulfonyl).

The aforementioned further substances arrange themselves when incorporated into a polar medium in the same way as the substances according to formulae (I, I* and Ia).

The composition according to the invention with active ingredient carriers which comprise aforementioned further substances comprises these usually to a molar fraction of at least 50%, preferably at least 70%, particularly preferably of 80 to 90%, based on the active ingredient carrier.

In preferred embodiments of the composition according to the invention, the active ingredient carrier comprises two lipids as further substances.

Within this preferred embodiment, one of the two lipids is preferably L-α-phosphatidylcholine. The L-α-phosphatidylcholine is particularly preferably the L-α-phosphatidylcholine which can be obtained from chicken eggs.

In a particularly preferred embodiment of the composition according to the invention, the active ingredient carrier comprises a molar fraction of L-α-phosphatidylcholine between 10 and 90% and a fraction of a substance according to formula (I, I* or Ia) based on the active ingredient carrier.

In a very particularly preferred embodiment of the composition according to the invention, the active ingredient carrier consists of a molar fraction of L-α-phosphatidylcholine between 10 and 30%, a molar fraction of 1,2-dioleoyl-sn-glycero-3-phosphoethanolamine between 50 and 65% and a remaining fraction of a substance according to formulae (I, I* or Ia), such that in total 100% molar fraction, based on the active ingredient carrier, are obtained.

The active ingredient carriers present in the composition according to the invention are liposomes, which comprise so-called lipid double layers, with the just-described hydrophilic chemical groups X of the substances according to formulae (I), (I*) (Ia), which are a constituent of this active ingredient carrier, orienting themselves to the polar solvent, whereas the hydrophobic chemical groups of the substances according to formulae (I), (I*) or of the above-described lipids of both layers are oriented into the interior of the lipid double layer.

The cytostatic agent present in the active ingredient carrier in the composition according to the invention is preferably one which is soluble in water or aqueous media to a concentration of up to 3 mmol/l.

Particularly preferred cytostatic agents are those selected from the list consisting of doxorubicin, pemetrexed, melphalan, amsacrin, asparaginase, bevacizumab, bleomycin, busulfan, irinotecan, carmustine, daunorubicin, cisplatin, dactinomycin, gancyclovir, cytarabine, dacarbazine, cytarabine, vindesine, oxaliplatin, cyclophosphamide, cetuximab, etoposide, epirubicin, fludarabine, 5-fluorouracil, gemcitabine, trastuzumab, ifosfamide, topotecan, cladribine, alemtuzumab, rituximab, methotrexate, mitomycin, mitoxantrone, gemtuzumab, carboplatin, aldesleukin, bendamustine hydrochloride, paclitaxel, docetaxel, thiotepa, arsenic trioxide, vinblastine, bortezomib, azacitidine, vincristine, vinorelbine, cidofovir and idarubicin.

Very particular preference is given to doxorubicin.

The present invention further provides a method for releasing a cytostatic agent from a composition comprising an active ingredient carrier in the form of a liposome, characterized in that amphiphilic substances according to formula (I, I* and/or Ia) present in the active ingredient carrier of the composition are cleaved at their disulfide group, into a hydrophobic moiety and a hydrophilic moiety.

The cleavage according to the method of the invention can be an enzymatically catalyzed chemical cleavage or can be a non-enzymatically catalyzed reduction in which the disulfide group of the substances according to formula (I, I* and/or Ia) is cleaved as a result of their reduction.

Preferably, the cleavage is an enzymatically catalyzed cleavage. The enzyme which catalyzes such a chemical cleavage is preferably a thioreductase, thiooxidase, thiooxireductase or thiol-disulfide oxidoreductase.

The non-enzymatically catalyzed cleavage of the disulfide group by reduction is likewise possible, in which case the reducing agent used is a substance selected from the list consisting of gluthathione, cysteine, tris(2-carboxyethyl)phosphine (TCEP), dithiothreitol (DTT) and 1,4-dithioerythritol (DTE). Preferably, the reducing agent is tris(2-carboxyethyl)phosphine (TCEP).

The aforementioned reducing agents are advantageous under certain circumstances because they usually have a reduction potential which merely suffices to reduce at least one of the two sulfur atoms of the disulfide group of the substances according to formula (I, I* and/or Ia), as a result of which its bond is cleaved. At the same time, however, the reduction potential of the aforementioned reducing agents does not suffice to reduce other chemical groups of the substances according to formula (I, I* and/or Ia) or groups of the further substances of the active ingredient carriers according to the invention in the form of a liposome.

Consequently, it is in particular ensured that a cleavage of the amphiphilic substances according to formula (I, I* and/or Ia) into a hydrophobic and a hydrophilic moiety takes place, as a result of which the release according to the invention takes place particularly advantageously.

Gluthathione and cysteine are substances which also occur in the body of mammals, in particular humans at inflammatorily altered sites in a particularly high concentration, as a result of which a targeted release is made possible particularly here with the present invention.

The method according to the invention for releasing a cytostatic agent is based on the surprising finding that it suffices to cleave the cytostatic agent according to formula (I, I* and/or Ia) present in the active ingredient carriers into a hydrophilic moiety and a hydrophobic moiety, which, without being bound to one theory, leads to the liposome being destabilized in such a way that it breaks open and releases the active ingredient present therein.

The use of the above-described compositions as medicament is further provided, particularly as a medicament for treating cancer.

The present invention is illustrated by reference to the examples and drawings below, but without limiting it thereto.

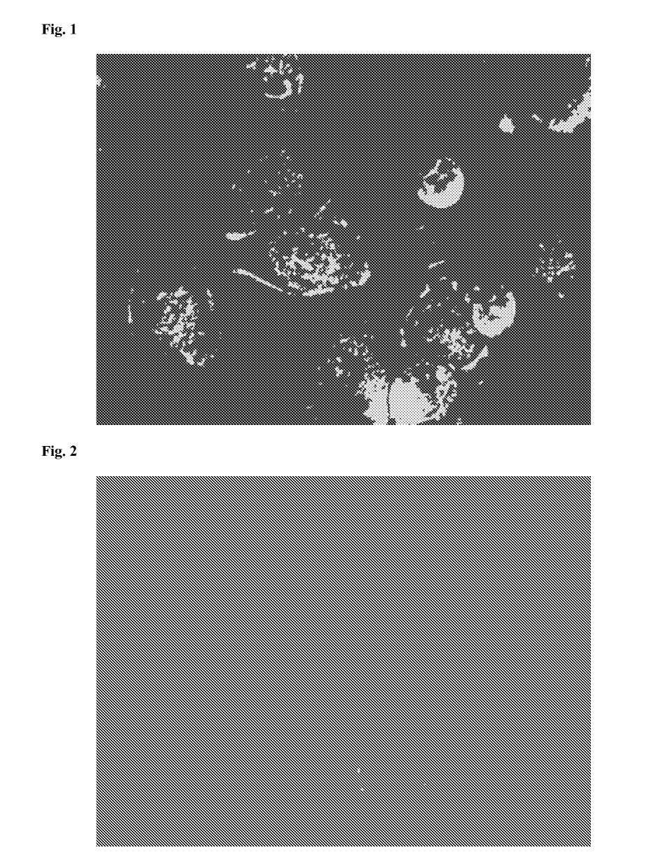

FIG. 1 shows an image of the cells which has been obtained by means of differential interference contrast, according to example 3.

FIG. 2 shows a fluorescence micrograph of the same cells as shown in FIG. 1.

The present invention is further illustrated by reference to the examples below without limiting it thereto.

### EXAMPLES

### Example 1

### Preparation of a Composition According to the Invention Comprising an Active Ingredient Carrier with a Substance According to Formula (Ia)

The substance according to formula (Ia) was prepared according to examples 1-4 of the international application PCT/EP2010/064272.

The substance according to formula (Ia), and also L-α-phosphatidylcholine (“eggPC”, from Avanti Polar Lipids, Alabaster, USA) and 1,2-dioleoyl-sn-glycero-3-phosphoethanolamine (“DOPE” from Avanti Polar Lipids, Alabaster, USA) were dissolved in chloroform/methanol (ratio 5:1).

The fractions of the aforementioned substances in the active ingredient carrier obtained later from this solution, based on the active ingredient carrier, are shown in Table 1.

TABLE 1
Composition of the active ingredient carrier of
the composition according to the invention
Molar fraction [%]

| Substance according | | |
|---|---|---|
| to formula (Ia) | “eggPC” | “DOPE” |
| 20 | 20 | 60 |

The resulting solution in the aforementioned chloroform/methanol mixture was subsequently treated in a rotary evaporator to the point of complete removal of the solvent, such that a composition according to Table 1 precipitated out as solid on the glass wall of the rotary evaporator in dry form as a film.

The precipitated film was then taken up in cyclohexane (in a mixture with 5% by volume ethanol) and the resulting solution was transferred to a glass tube which was frozen at −80° C. and lyophilized overnight.

Afterwards, an aqueous doxorubicin-hydrochloride solution (1 ml with a concentration of 0.5 mg/ml) was added to the dry composition and the mixture was treated with ultrasound for 10 minutes. The suspension obtained in this way was subjected to five freeze-thaw processes, thawing taking place in each case using a 70° C.-hot water bath. Then, firstly 0.1 ml and, after 5 minutes, a further 0.4 ml of aqueous doxorubicin solution (in each case with a concentration of 0.5 mg/ml) were added to the suspension. Consequently, an end concentration of ˜3 mmol/l doxorubicin hydrochloride could be achieved.

The suspension was then passed 11 times through a polycarbonate filter with a pore diameter of 100 nm.

This gave the composition according to the invention with doxorubicin hydrochloride as cytostatic agent with an active ingredient carrier comprising a substance according to formula (Ia), with unilamellar vesicles (active ingredient carriers) which are laden with doxorubicin hydrochloride being formed in the aforementioned aqueous solution.

In order to free the aforementioned composition from excess doxorubicin hydrochloride located in the aqueous solution and which is not located in the interior of the active ingredient carriers, the permeate from the aforementioned filtration was passed over a gel filtration column located in a centrifuge unit (column: Sephadex G 50 (Fine, Sigma-Aldrich) in 1 ml polypropylene columns, Qiagen; centrifugation: 3 minutes at 100-300×g).

### Example 2

### Functionalization of the Composition with Monoclonal Antibodies

150 μl of an aqueous solution comprising the composition according to example 1 and 25 μl of an aqueous streptavidin solution (1 mg/ml) were added to a PBS (phosphate buffered saline) buffer solution without calcium and magnesium ions such that a total volume of 1 ml was achieved.

This mixture was left to stand at room temperature for 30 minutes.

Afterwards, 50 μl of anti-human-p185HER2 biotin conjugate (Invitrogen, Karslruhe, used as acquired) were added and the mixture was incubated at 4° C. for one hour.

This gave a composition which comprises active ingredient carriers composed according to example 1 comprising the cytostatic agent doxorubicin hydrochloride and where these active ingredient carriers have on their outer shell anti-human-p185HER2 antibodies which are known to specifically bind specially to human p185HER2 proteins located on the surface of human breast cancer cells.

### Example 3

### Cell Culture Tests with Composition from Example 2

Human breast cancer cells of the type BT-474 (DSMZ strain No. ACC 64) were cultured in a 12 ml cell culture flask as monolayer up to a confluency of approx. 70%. The medium used was DMEM with 10% FBS, 5% penicillin-streptomycin and 2 mM L-glutamine.

One day prior to microscope analysis, the cells were removed from the bottom of the culture flask with the help of a 0.05% strength trypsin/EDTA solution and transferred to a 35 mm glass-bottomed vessel (DMEM, 1:4 dilution).

The cells were incubated at 37° C. and 5% CO₂ and treated with 1 ml of a PBS solution which comprised 25 μl of the composition from example 2.

The result of this exposure experiment was evaluated by fluorescence microscopy (inverted epifluorescence microscope, Zeiss, model: Axiovert 100). Prior to each micrograph, the cells were washed four times with PBS with calcium and magnesium ions.

For each evaluation, three micrographs were recorded and the fluorescence intensity was determined in each case at ten points within the cells and the average fluorescence was calculated therefrom. The procedure was carried out analogously with three times ten positions outside of the cells in order to ascertain the average background fluorescence. The difference therefrom gives the average fluorescence signal.

Since the cytostatic agent doxorubicin hydrochloride is known to be fluorescence-active it was therefore possible to ascertain whether the cytostatic agent was released and absorbed by the cells.

It was found here that this average fluorescence signal (difference between background and fluorescence of the cells by the active ingredient) was 58.94±11.17 (a.u.). This is sufficient to demonstrate the absorption and thus the preceding release of the cytostatic agent.

FIG. 2 shows for this again separately the selective fluorescence signal in the cells, the light areas illustrating the fluorescence signals of the doxorubicin in the cells (as shown in FIG. 1 in differential interference contrast photograph). These would be recognizable in terms of color as red-orange light emission. For each cell according to FIG. 1 a corresponding fluorescence signal is recognized in the interior of the cells, such that the absorption by the cells and thus the prior release of the doxorubicin hydrochloride is detected. Since no reducing agent has been added to the cell culture, the release from the composition according to the invention took place merely by virtue of the presence of the cells.

1.-10. (canceled)

11. A composition comprising an active ingredient carrier in the form of a liposome which comprises a cytostatic agent, characterized in that the active ingredient carrier comprises at least one substance according to formula (I) 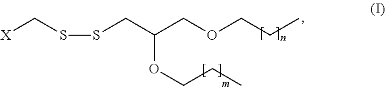
where the at least one substance according to formula (I)
(a) has in X a chemical group comprising at least five carbon atoms and comprising at least one ether group and/or amine group or ammonium group, and where
(b) n and m, independently of the other, is a natural number from 1 to 30, but where the sum of n and m is at least 16.

12. The composition as claimed in claim 11, characterized in that the chemical group X comprises at least eight carbon atoms.

13. The composition as claimed in claim 11, characterized in that the chemical group X additionally comprises at least one carboxylic acid ester group.

14. The composition as claimed in claim 11, characterized in that the at least one amine group or ammonium group of the chemical group X of the substances according to formula (I) in the active ingredient carrier is a quaternary ammonium group.

15. The composition as claimed in claim 11, characterized in that the active ingredient carrier comprises a substance according to formula (I*) 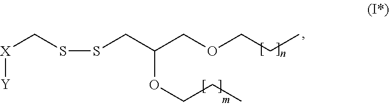
where the chemical group Y is a chemical group selected from the group consisting of biotin, protein, peptide, glyco group, nucleic acid, nucleoside and nucleotide.

16. The composition as claimed in claim 11, characterized in that the active ingredient carrier comprises a substance according to formula (Ia)

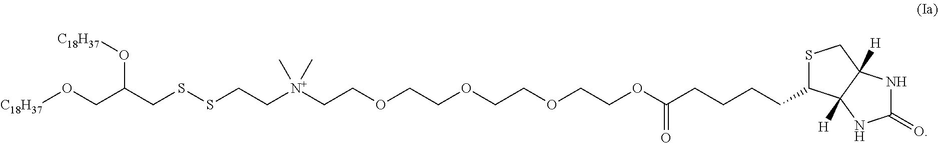

17. The composition as claimed in claim 11, characterized in that it comprises, in the active ingredient carrier, also phospholipids.

18. The composition as claimed in claim 11, characterized in that the cytostatic agent is selected from the group consisting of doxorubicin, pemetrexed, melphalan, amsacrin, asparaginase, bevacizumab, bleomycin, busulfan, irinotecan, carmustine, daunorubicin, cisplatin, dactinomycin, gancyclovir, cytarabine, dacarbazine, cytarabine, vindesine, oxaliplatin, cyclophosphamide, cetuximab, etoposide, epirubicin, fludarabine, 5-fluorouracil, gemcitabine, trastuzumab, ifosfamide, topotecan, cladribine, alemtuzumab, rituximab, methotrexate, mitomycin, mitoxantrone, gemtuzumab, carboplatin, aldesleukin, bendamustine hydrochloride, paclitaxel, docetaxel, thiotepa, arsenic trioxide, vinblastine, bortezomib, azacitidine, vincristine, vinorelbine, cidofovir and idarubicin.

19. A method for releasing a cytostatic agent from a composition comprising an active ingredient carrier in the form of a liposome as claimed in claim 11, characterized in that amphiphilic substances according to formula (I) present in the active ingredient carrier of the composition are cleaved at their disulfide group, into a hydrophobic moiety and a hydrophilic moiety.

20. A medicament comprising the composition as claimed in claim 11.

21. The composition as claimed in claim 11, characterized in that it comprises, in the active ingredient carrier, phosphatidylcholine and/or phosphatidylethanolamine.

22. A method for releasing a cytostatic agent from a composition comprising an active ingredient carrier in the form of a liposome as claimed in claim 15, characterized in that amphiphilic substances according to formula (I*) present in the active ingredient carrier of the composition are cleaved at their disulfide group, into a hydrophobic moiety and a hydrophilic moiety.

23. A method for releasing a cytostatic agent from a composition comprising an active ingredient carrier in the form of a liposome as claimed in claim 16, characterized in that amphiphilic substances according to formula (Ia) present in the active ingredient carrier of the composition are cleaved at their disulfide group, into a hydrophobic moiety and a hydrophilic moiety.
